(19) United States
(12) Patent Application Publication
(10) Pub. No.: US 2021/0353573 A1
(43) Pub. Date: Nov. 18, 2021

(54) A SOFT-GEL CAPSULE FORMULATION, METHOD OF MANUFACTURE AND USE THEREOF

(71) Applicant: Chanelle Pharmaceuticals Manufacturing Ltd., Loughrea (IE)

(72) Inventors: Michael Hilary BURKE, Loughrea (IE); Helena Maria de Albuquerque Ferreira TELES, Tilburg (NL); Martin PIEST, Tilburg (NL)

(21) Appl. No.: 17/283,657

(22) PCT Filed: Oct. 3, 2019
(86) PCT No.: PCT/EP2019/076821

§ 371 Date: Apr. 8, 2021

(30) Foreign Application Priority Data
Oct. 8, 2018 (IN) 201821038030
Jul. 8, 2019 (GB) 1909784.9

(51) Int. Cl. A61K 31/192; A61K 31/198; A61K 31/23; A61K 9/48; A61K 47/24; A61K 9/00

(52) U.S. Cl. CPC A61K 31/192; A61K 31/198; A61K 31/23; A61K 9/4833; A61K 47/24; A61K 9/4825; A61K 9/0053; A61K 9/4858

(57) ABSTRACT

The present invention is directed to a suspension-based liquid fill formulation for a capsule comprising ibuprofen lysine and a medium chain triglyceride (MCT). More particularly, the present invention is directed to a soft-gel capsule containing suspension-based fill formulation ibuprofen lysine, medium chain triglyceride (MCT) and lecithin, method of manufacture and use thereof.

### CROSS-REFERENCE TO RELATED APPLICATIONS

This application is the U.S. National phase application corresponding to PCT/EP2019/076821 which was assigned an international filing date of Oct. 3, 2019 and associated with publication WO 2020/074361 A1 and which claims priority to GB patent application 1909784.9, filed Jul. 8, 2019 and IN patent application 201821038030, filed Oct. 8, 2018, the disclosures of all these applications are expressly incorporated herein by reference.

### FIELD OF THE INVENTION

The present invention generally relates to the field of pharmaceutical sciences and to a suspension-based liquid fill formulation for a capsule comprising ibuprofen lysine and a medium chain triglyceride (MCT). More particularly, the present invention relates to a soft-gel capsule fill formulation comprising ibuprofen lysine, medium chain triglyceride (MCT) and lecithin, a method of manufacture and use thereof.

### BACKGROUND OF THE INVENTION

Ibuprofen, namely 2-(4-isobutylphenyl) propionic acid, is a well-known analgesic, anti-inflammatory and anti-pyretic agent. The acid form of ibuprofen has low solubility, which is overcome using lysine salt of ibuprofen. Following oral administration, ibuprofen lysine dissociates to ibuprofen acid and lysine. Lysine has no recognized pharmacological activity. Ibuprofen is more rapidly adsorbed from the GI tract though the rate of extent of ibuprofen absorption may reduce by food. Most preparations contain racemic mixture of R-(−)- and S-(+)-ibuprofen, with the S-(+) enantiomer possessing the majority of pharmacological activity. U.S. Pat. No. 4,279,926 describes the use of lysine salt of ibuprofen is relieving pain and inflammatory conditions in warm-blooded animals.

Ibuprofen lysine is currently available in the form of tablets and caplets marketed as Nurofen® by Reckitt-Benckiser. However, there are no liquid filled formulations/capsules, including soft-gel formulations or two-piece hardgels of ibuprofen lysine available in the market.

Soft capsules in particular, have gained popularity and acceptance over tablets due to their elegant and clear appearance. Soft capsules are in general easier to swallow, conceal unpleasant odours, are hermetically sealed, and can easily be coloured to protect ingredients from light. Additionally, in general soft capsules often allow for a faster onset of action as the active ingredient is already in a liquid form (solubilized or dispersed) which tends to speed up absorption.

European Patent No. 1321140 describes a liquid fill composition containing ibuprofen, polyethylene glycol, water and terpenoid. Another European Patent No. 1485081 teaches liquid fill composition of ibuprofen for hard-shell capsules whereas a soft-gel capsule of ibuprofen comprising polyoxyethylene-polyoxypropylene-diol and surfactant as fill formulation is disclosed in the European Patent No. 178436. None of these patent literatures suggests use of lysine salt of ibuprofen. However, these liquid fill formulations do not overcome the inherent solubility problems of ibuprofen.

Thus, there is an unmet need for liquid-fill formulations, particularly a soft-gel formulation of ibuprofen lysine, which is both commercially viable and therapeutically stable.

### SUMMARY OF THE INVENTION

The present invention provides a suspension-based liquid-fill formulation, preferably a soft-gel capsule formulation, of ibuprofen lysine. The formulation is a non-conventional suspension-based liquid fill formulation which is subsequently encapsulated.

According to a general aspect of the invention, there is provided a suspension-based liquid fill formulation for a capsule comprising ibuprofen lysine and a medium chain triglyceride (MCT).

More particularly, according to a first aspect of the invention, there is provided a suspension-based liquid fill formulation comprising ibuprofen lysine, triglyceride, preferably medium chain triglyceride (MCT), and lecithin. Preferably, the ibuprofen lysine has a d50 value of less than 15 μm and a d90 value more than 10 μm.

According to a second aspect of the invention, there is provided an orally administrable soft-gel capsule comprising the suspension-based fill formulation of any of the preceding claims. According to a preferred embodiment, the soft-gel capsule comprises gelatin, plasticizer and purified water.

According to a third aspect of the invention, there is provided a method of preparing a suspension-based fill formulation for soft-gel capsule, comprising the steps of:
preparing a mixture of medium chain triglyceride (MCT) and lecithin; and adding ibuprofen lysine to the mixture,
wherein the ibuprofen lysine having a d50 value of less than 15 μm and a d90 value more than 10 μm.

According to a fourth aspect of the invention, there is provided a soft-gel capsule of the invention for use in the treatment of a headache or migraine.

According to a fifth aspect of the invention, there is provided a method for the treatment of a headache or migraine comprising the step of administering a therapeutically effective amount of the soft-gel capsule according to the invention to a subject in need thereof.

This invention, including its embodiments and other features, will become more apparent from the following detailed description and non-limiting examples.

### DETAILED DESCRIPTION OF THE INVENTION

In this specification, “Ibuprofen” as referred herein refers to ibuprofen lysine, and/or its enantiomers, polymorphs, solvates, hydrates and mixtures thereof. Additionally, ibuprofen lysine (the lysine salt of ibuprofen) can be referred to interchangeable as “ibuprofen lysinate”.

In this specification, it will be understood that ‘a soft-gel capsule fill formulation’ is a softgel or soft gelatin capsule comprising a solid capsule (outer shell) surrounding a liquid or semi-solid centre (inner fill) with active agent (in this case ibuprofen lysine). It will be understood that the active ingredient may be incorporated into the outer shell and/or the inner fill. The soft-gel capsules of the invention are oral dosage forms similar to conventional tablets or caplets. Conventional softgel shells are typically made from a combination of gelatin, water, opacifier and/or a plasticizer such as glycerin and/or sorbitol.

In this specification, the term ‘medium chain triglyceride’ are triglycerides with two or three fatty acids having an aliphatic tail of 6-12 carbon atoms. As defined in European Pharmacopoeia 9th Edition (Ph. Eur) 6.2, MCTs typically consist of a mixture of triglycerides of saturated fatty acids, mainly of octanoic acid (Cs) and of capric acid (C₁₀) (C₁₀H₂₀O₂=172.3). Ideally, they contain not less than 95% of saturated fatty acids with 8 and 10 carbon atoms. The metabolism of MCT differs from the more common long chain triglycerides (LCT) due to the physical properties of MCT and their corresponding medium chain fatty acids (MCFA). Due to the short chain length of MCFA, they have lower melting temperatures, for example the melting point of MCFA (C8:0) is 16.7° C., compared with 61.1° C. for the LCFA (C16:0). Hence, MCT and MCFA are liquid at room temperature. MCT are highly ionized at physiological pH, thus they have much greater solubility in aqueous solutions than LCT. The enhanced solubility and small size of MCT also increases the rate at which fine emulsion particles are formed.

The term “bioequivalence” as used herein denotes a scientific basis on which two or more pharmaceutical compositions containing same active ingredients are compared with one another. “Bioequivalence” means the absence of a significant difference in the rate and extent to which the active agent in pharmaceutical equivalents or pharmaceutical alternatives becomes available at the site of action when administered in an appropriately designed study. Bioequivalence can be determined by an in vivo study comparing a pharmacokinetic parameter for the two compositions. Parameters often used in bioequivalence studies are Tₘₐₓ, Cₘₐₓ, AUC_{0-∞}, AUC₀₋ₜ.

In this specification, the term “bioequivalence” is primarily based on the pharmacokinetic parameters AUC₀₋ₜ and Cₘₐₓ derived from S-(+)-ibuprofen concentrations determined in individual plasma samples. The term “Cₘₐₓ” denotes the maximum plasma concentration; “Tₘₐₓ” denotes the time to reach the Cₘₐₓ after administration; AU_{0-inf} or AUC denotes the area under the plasma concentration versus time curve from time 0 to infinity; AUC₀₋ₜ denotes the area under the plasma concentration versus time curve from time 0 to time t. For statistical analysis of pharmacokinetic data, the logarithmic transformed AUC₀₋ₜ, AUC_{0-∞}, or Cₘₐₓ data can be analysed statistically using analysis of variance.

After considerable experimentations and meticulous studies, the inventors found that conventional solution formulations were not feasibly viable as they could not provide soft-gels with an acceptable size. Accordingly, the inventors developed a non-conventional soft-gel capsule formulation containing suspension-based fill formulations to provide desirable characteristics.

A variety of different carrier materials for suspension of ibuprofen lysine were tested during the development phase. Unexpectedly, the conventional choice, polyethylene glycol (PEG) based suspension formulations were found to be too sticky and did not have desirable flow characteristics.

However, it has now been advantageously found that the combination of medium chain triglycerides (MCT) oil and lecithin provides a soft-gel capsule of ibuprofen lysine with desirable properties. It was found that addition of various conventional stabilizers like silica, hydrogenated soyabean oil, gelucire, beeswax or vitamin E TPGS to the MCT oil and lecithin mixture did not significantly improve the dissolution and stability characteristics of the formulation.

According to a preferred embodiment, the suspension-based fill formulation comprises ibuprofen lysine:MCT from 1:1 to 1:2 by weight, preferably from 1:1 to 1:1.5 by weight.

According to a still further embodiment, the suspension-based fill formulation comprises from about 30-70% by weight of MCT, from about 30-60% by weight of ibuprofen lysine and from about 0.1-10%, preferably 1-10%, by weight lecithin.

The soft-gel capsule formulation of ibuprofen lysine of the present invention has been designed to be bioequivalent to the commercially available Nurofen® caplets.

Additionally, in order to be acceptable to patients, the soft-gel capsule should be of size that can easily be swallowed.

In accordance to the invention, ibuprofen lysine is ideally in powdered form having the particle size distribution such that 50% (D₅₀) of particles are of 5 μm to 15 μm, preferably 6 μm to 10 μm, and 90% (D₉₀) of particles are of 10 to 29 μm, preferably 13 μm to 28 μm.

The “particle size” of a particle to be determined is understood for the purposes of the invention to mean the diameter of an equivalent particle which is assumed to be spherical and to have the same light-scattering pattern as the particles to be determined. In this application, the particle size was determined by using Malvern Instruments Mastersizer.

The D₉₀ value of the particle size distribution describes the particle size at which 90% by volume of the particles have a smaller particle size than the particle size corresponding to the D₉₀ value.

The D₅₀ value of the particle size distribution describes the particle size at which 50% by volume of the particles have a smaller particle size and remaining 50% have a larger particle size than the particle size corresponding to the D₅₀ value.

The D₁₀ value of the particle size distribution describes the particle size at which 10% by volume of the particles have a smaller particle size than the particle size corresponding to the D₁₀ value.

In a preferred embodiment, the powdered ibuprofen lysine has particles with a D₁₀ value of 3 μm to 3.8 μm, preferably 3.1 μm to 3.7 μm.

According to a second aspect of the invention, there is provided a method of preparing a suspension-based fill formulation for soft-gel capsule, comprising the steps of:
preparing a mixture of medium chain triglyceride (MCT) and lecithin; and adding ibuprofen lysine to the mixture,
wherein the ibuprofen lysine having a d₅₀ value of less than 15 μm and a d₉₀ value more than 10 μm.

Optionally, the method further comprises a step of heating the mixture before step (ii), preferably to a temperature above 50° C.

Preferably, the suspension-based fill formulation is homogenised by adding a colloid mill.

For example, the fill formulation for encapsulation may ideally be prepared by mixing MCT and lecithin in a dissolver and then adding ibuprofen lysine to the mixture. Heating the mixture to 55±5° C. prior to addition of ibuprofen lysine provides excellent dispersion properties. Also, setting the mixing time to 30 minute ensures homogeneity of the mixture. The fill is homogenized using a colloid mill. It is found that wetting and sedimentation was positively affected by the colloid mill treatment. The fill formulation is de-aerated prior to encapsulation and mixture cooled and temperature controlled at 32±3° C.

In contrast to known ibuprofen liquid based formulations, the fill formulation advantageously has shear-thinning behaviour, meaning the material is more viscous at low shear than at high shear. This is desired for a soft-gel suspension fill as a high viscosity at low shear slows down sedimentation of the medicine upon standing and a low viscosity at high shear ensures good flow properties upon mixing and pumping and allows a suitable machine speed for encapsulation. Thus, the inventors have advantageously utilised the shear thinning behaviour of the fill formulation of the invention to aid the soft-gel encapsulation processing and resultant end product.

The main ingredients of the soft-gel capsule shell of the present invention include gelatin, plasticizer, and purified water. The shell plasticizer preferably is sorbitol. While glycerol can be used as a plasticizer, it has been found that the ibuprofen lysine turned brownish in the presence of glycerol indicating degradation. Therefore, the non-glycerin plasticizers are also preferred. The ingredients are combined to form molten gel mass that forms the ribbons in the soft-gel encapsulation process.

Encapsulation of the soft-gel capsules may be done in a known manner with a rotary die process in which a molten mass of a gelatin shell formulation is fed from onto drums to form two ribbons of gelatin in a semi-molten state. These ribbons travel between pair of rotating dies while the medicine is delivered through the wedge to the die cavities. The dies press together, and the combination of heat and pressure causes the left and right gel ribbons to fuse around the medicine and form the capsule.

The soft-gels are dried in a soft-gel (tumbling) drier and the dry soft-gels are inspected, polished, sized, printed and metal checked.

The inventors advantageously found that the soft-gel capsule as defined herein is bioequivalent to Nurofen® Caplet and advantageously the bioequivalence of the soft-gel capsule is established by: (a) a 90% Confidence Interval for AUC₀₋ₜ between 0.8586 and 0.9599, and (b) a 90% Confidence Interval for Cₘₐₓ between 0.9670 and 1.0985 under fasting conditions.

According to another embodiment of the present invention, there is provided a soft-gel capsule as defined herein for use in the treatment of a headache or a migraine.

According to yet another embodiment of the present invention, there is provided the use of a soft-gel capsule as defined herein for the manufacture of a medicament for the treatment of a headache or a migraine.

According to a still further embodiment of the present invention, there is provided a method for the treatment of a headache or migraine comprising the step of administering a therapeutically effective amount of the soft-gel capsule as defined herein to a subject in need thereof.

The present invention is further illustrated by the following examples which are provided merely to be exemplary of the invention and do not limit the scope of the invention. Certain modifications and equivalents will be apparent to those skilled in the art and are intended to be within the scope of the invention.

The invention will now be described by the following non-limiting figures and examples/comparative examples.

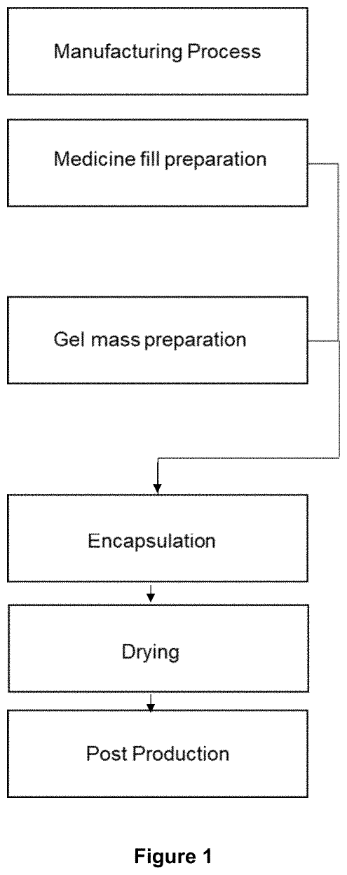

FIG. 1 is a flowchart of the manufacturing process steps 1 to 5.

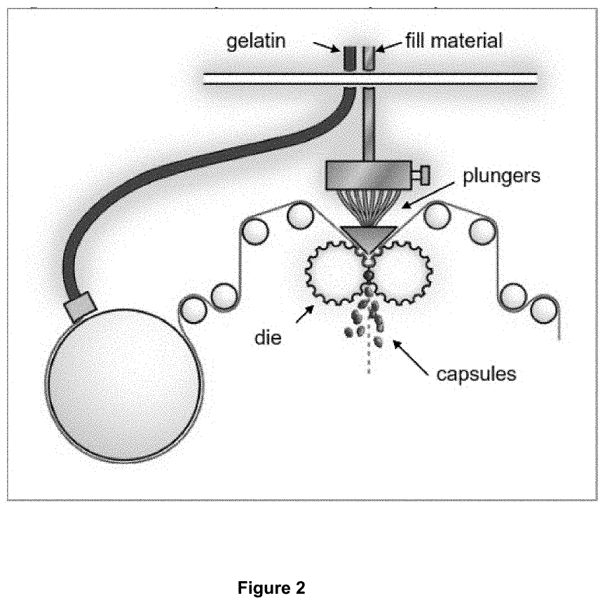

FIG. 2 is a schematic representation of the preferred rotary die encapsulation, a conventional soft-gel encapsulation using the fill formulation of step 1 and gel mass of step 2).

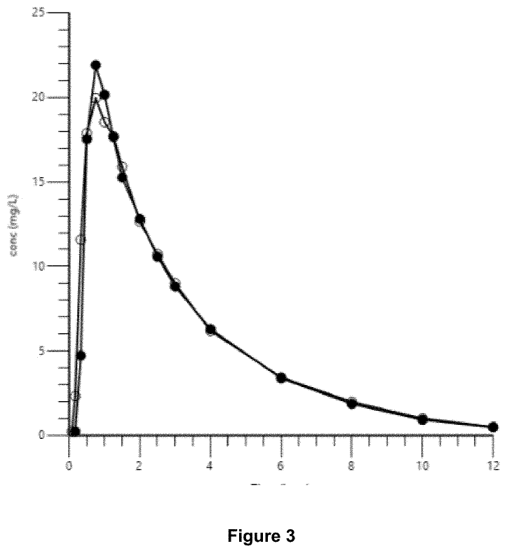

FIG. 3 illustrates geometric means of plasma S-(+)-ibuprofen concentration (mg/L) versus time curves following a single 684 mg dose of ibuprofen lysine in 25 subjects.

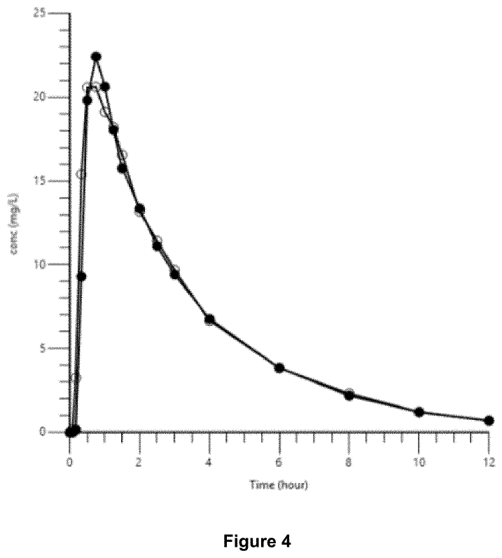

FIG. 4 illustrates arithmetic means of plasma S-(+)-ibuprofen concentration (mg/L) versus time curves following a single 684 mg dose of ibuprofen lysine in 25 subjects.

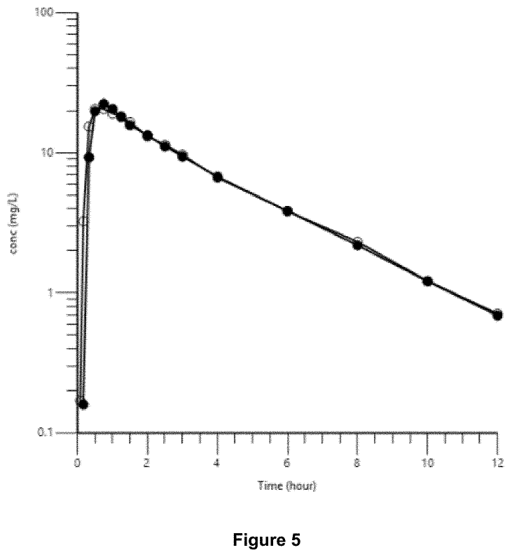

FIG. 5 illustrates semilogarithmic plot of arithmetic means of plasma S-(+)-ibuprofen concentration (mg/L) versus time curves following a single 684 mg dose of ibuprofen lysine in 25 subjects.

The manufacturing process FIG. 1 comprises steps 1 to 5, as described below.

Step 1: Medicine Fill Preparation

MCT and lecithin are mixed and heated to above 50° C. in a dissolver. Once mixed, ibuprofen lysine is added and mixed under high shear conditions. Once a homogenous mixture is obtained, the mixture is further homogenized using a colloid mill to in to break any agglomerates and achieve the desired particle size—typically until the suspension has a particle size of NMT 150 um when tested using a Hegman gauge. The fill formulation is placed in a container and de-aerated.

Step 2: Gel Mass Preparation

Conventional gel mass preparation techniques may be utilized. For example, the main ingredients of a softgel capsule shell are gelatin, plasticizer (such as sorbitol or a non-glycerin plasticizer), and purified water. Typical gel formulations contain (w/w) from about 40-50% gelatin, about 20-30% plasticizer, and about 30-40% purified water. Most of the water is subsequently lost during capsule drying. The ingredients are combined to form a molten gelatin mass using either a cold melt or a hot melt process. The prepared gel masses are transferred to preheated, temperature-controlled, jacketed holding tanks where the gel mass is aged at 50-60° C. until used for encapsulation.

Cold Melt Process

The cold melt process generally involves mixing gelatin with plasticizer and chilled water and then transferring the mixture to a jacket-heated tank. Typically, gelatin is added to the plasticizer at ambient temperature (18-22° C.). The mixture is cooked (57-95° C.) under vacuum for 15-30 minutes to a homogeneous, deaerated gel mass. Additional shell additives can be added to the gel mass at any point during the gel manufacturing process or they may be incorporated into the finished gel mass using a high torque mixer.

Hot Melt Process

The hot melt process generally involves adding, under mild agitation, the gelatin to a preheated (60-80° C.) mixture of plasticizer and water and stirring the blend until complete melting is achieved. While the hot melt process is faster than the cold melt process, it is less accurately controlled and more susceptible to foaming and dusting.

Step 3: Encapsulation (See FIG. 2)

Softgel Capsule

Softgel capsules are typically produced using a rotary die encapsulation process. The gel mass is fed either by gravity or through positive displacement pumping to two heated (48-65° C.) metering devices. The metering devices control the flow of gel into cooled (10-18° C.), rotating casting drums. Ribbons are formed as the cast gel masses set on contact with the surface of the drums.

The ribbons are fed through a series of guide rolls and between injection wedges and the capsule-forming dies. A food-grade lubricant oil is applied onto the ribbons to reduce their tackiness and facilitate their transfer. Suitable lubricants include mineral oil, medium chain triglycerides, and soybean oil. Fill formulations are fed into the encapsulation machine by gravity. In the preferred embodiment, the softgels contain printing on the surface, optionally identifying the encapsulated agent and/or dosage

For example, according to a preferred embodiment, the fill material and the gel mass are processed into soft gelatin capsules using the rotary die method. During encapsulation the ribbon is lubricated on both sides with medium chain triglycerides (MCT) and the outer side of the capsule is lubricated also with medium chain triglycerides/lecithin. The fill material is pre-heated (approximately 30±5° C.) and mixed prior and during encapsulation and the process is set to deliver the required amount of fill material. The fill weights are tested for uniformity and quantity by taking capsules from a series of plungers. During manufacturing the Ibuprofen lysinate content of the capsules is controlled by fill weight check.

Steps 4 and 5 are conventional steps.

Step 4: Drying

Step 5: Post-production (i.e. packaging according to conventional means).

### Comparative Example 1

A suspension-based formulation was investigated using PEG or MCT oil as the main suspending vehicle.

PEG 400 and PEG 600 were pre-heated to 40° C. and ibuprofen lysinate was added slowly to the PEG whilst maintaining the temperature at 40° C. However, both formulations became too dry during this step and even after the addition of water no improvement was observed.

It was found that an MCT oil-based suspension had the highest potential for developing a commercially viable formulation.

Various excipients with surfactant and/or stabilizer properties were also tested in terms of dissolution profiles. These included Vitamin E TPGS, silica, hydrogenated soybean oil. However, they did not show significant dissolution profiles and were not suitable for a softgel manufacturing process.

### Comparative Example 2

Various studies were carried out to determine an appropriate capsule shell formulation. The use of a glycerol-based gel formulation was excluded after excipient compatibility studies showed that mixtures of glycerol and ibuprofen lysinate turned brownish indicating possible chemical reaction, e.g. degradation, between the excipient and API.

### Example 1

Soft-Gel Capsule Fill Formulation

Ibuprofen lysine soft-gel capsules having strength of 342 mg (corresponding to 200 mg Ibuprofen) and 684 mg (corresponding to 400 mg Ibuprofen) as shown in Tables 1 (fill formulation) and capsule dry shell were prepared according to the method of Example 2.

TABLE 1
fill formulation

| | mg/capsule | Mg/capsule | |
|---|---|---|---|
| | (342 mg | (684 mg | |
| Description | strength) | strength) | Function |
| Ibuprofen lysine | 342 | 684 | API |
| Medium chain | 375 | 750 | Suspending |
| triglycerides (MCT) | | | Agent |
| Lecithin (soybean) | 12 | 24 | Emulsifier |

Capsule shell formulations contain (w/w) 40-50% gelatin, 20-30% plasticizer (e.g. sorbitol, partially dehydrated), and 30-40% purified water. Colouring agent may be added.

### Example 2

Manufacturing process For a batch of 100,000 capsules, capsule shell was prepared according to table 2 and stored at 56±2° C. for 8-72 hours prior to encapsulation.

The fill formulation excipients were transferred to a medicine container and mixed until homogenous with Kreiss dissolver while heating to 55±5° C. API (i.e. Ibuprofen lysine) was added and mixed till homogenous suspension was formed (optimum mixing time set at 30 minutes). After this, the mixture was circulated through colloid mill for homogenization and then transferred to clean container for de-aeration. Temperature of the fill formulation was brought down to 32±3° C. during staging prior to encapsulation.

Encapsulation was performed according to methods known to person skilled in the art as illustrated in FIG. 2. The bulk product was packed in a double polyethylene bag placed in a carton box ready for finished product packaging into a conventional blister pack.

Results:

The stability in terms of shelf-life specifications of Ibuprofen Lysinate 342 mg and 684 mg soft capsules were tested using standard procedures and found to comply with the USP/Ph. EUr/ICH quality standards. Result are shown in Table 2 below.

TABLE 2

| | Acceptance criteria | | |
|---|---|---|---|
| | | At the end of | |
| Test | At release | shelf life | Procedure |
| Appearance | Complies at release and after 36 months | | Visual evaluation |
| Uniformity of dosage units | Complies | Complies | Ph. Eur. 2.9.40 test for |
| | | | content uniformity |
| Disintegration | Complies | Complies- | Ph. Eur. 2.9.1 |
| Dissolution (HPLC) | 75% (Q) in 30 min | | Ph. Eur. 2.9.3 and |
| | | | In-house |
| Identification of Ibuprofen: | Complies | Complies- | |
| Assay of Ibuprofen | Complies | | |
| (HPLC) | | | |
| Degradation Products | Complies | Complies | |
| Ibuprofen): | | | |

| | | |
|---|---|---|
| Total aerobic microbial | Complies | Ph. Eur. 2.6.13 |
| count (TAMC) | | Ph. Eur. 2.6.13 |
| Total combined | | Ph. Eur. 2.6.12 |
| yeasts/moulds count | | |
| (TYMC) | | |
| E. coli | | |

### Example 3: Bioequivalence Studies

In a bioequivalence study, the comparison of relative bioavailability of ibuprofen soft-gel capsule prepared according to examples 1 and 2 and Nurofen® Caplets was carried out in 26 healthy volunteers under fasted conditions. In this case, “fasted” is based on a 10-hour absence of food.

Results of bioequivalence studies of soft-gel capsule prepared according to example 1 and Nurofen® Caplet under fasting conditions is as indicated in the Table 3 below:

TABLE 3
Results of bioequivalence Studies
Under Fasting Condition (N = 25)

| Ibuprofen soft-gel capsule | | | Nurofen ® Caplet | | |
|---|---|---|---|---|---|
| AUC₀₋ₜ | Cₘₐₓ | tₘₐₓ | AUC₀₋ₜ | Cₘₐₓ | tₘₐₓ |
| (ng · h/mL) | (ng/mL) | (hours) | (ng · h/mL) | (ng/mL) | (hours) |

Geometric Mean

| | | | | | |
|---|---|---|---|---|---|
| 67.87 | 24.50 | NC | 69.37 | 23.78 | NC |

Arithmetic Mean

| | | | | | |
|---|---|---|---|---|---|
| 71.56 | 25.07 | 0.66 | 73.00 | 24.43 | 0.70 |

Standard Deviation

| | | | | | |
|---|---|---|---|---|---|
| 26.25 | 5.48 | 0.19 | 25.88 | 5.86 | 0.35 |

Coefficient of Variation (% CV)

| | | | | | |
|---|---|---|---|---|---|
| 36.7 | 21.9 | 29.2 | 35.5 | 24.0 | 49.8 |

Statistical Parameters

| Parameters | Cₘₐₓ | AUC₀₋ₜ |
|---|---|---|
| Geometric Mean Ratio | 1.0306 | 0.9078 |
| Lower Limit of 90% | 0.9670 | 0.8586 |
| Confidence Interval | | |
| Upper Limit of 90% | 1.0985 | 0.9599 |
| Confidence Interval | | |

Results of Table 4 demonstrate set of pharmacokinetic parameters AUC₀₋ₜ, Cₘₐₓ, Tₘₐₓ in 25 subjects for ibuprofen soft-gel capsule prepared according to examples 1 and 2 compared to Nurofen® Caplets.

The geometric mean ratio and the lower and upper limit of confidence interval for the bioequivalence extent parameters Cₘₐₓ and AUC₀₋ₜ were found well within the bioequivalence acceptance interval 0.8000 and 1.2500.

The specification and the examples are to be regarded in an illustrative rather than in a restrictive sense.

1. A suspension-based liquid fill formulation for a capsule comprising ibuprofen lysine and a medium chain triglyceride (MCT).

2. The suspension-based fill formulation of claim 1 for a soft-gel capsule comprising ibuprofen lysine and a medium chain triglyceride (MCT),
wherein the ibuprofen lysine has a d₅₀ value of less than 15 μm and a d₉₀ value more than 10 μm.

3. The suspension-based fill formulation of claim 2 wherein ibuprofen lysine:MCT is present from 1:1 to 1:2 by weight, preferably from 1:1 to 1:1.5 by weight.

4. The suspension-based fill formulation of claim 3, further comprising lecithin.

5. The suspension-based fill formulation of claim 4 comprising from about 40-70% by weight of MCT, from about 30-60% by weight of ibuprofen lysine and from about 1-10% by weight lecithin.

6. The suspension-based fill formulation of claim 1 wherein ibuprofen lysine is in powdered form having a particle size distribution such that 50% (D₅₀) of particles are of 5 μm to 15 μm, preferably 6 μm to 10 μm; and 90% (D₉₀) of particles are of 10 to 29 μm, preferably 13 μm to 28 μm.

7. An orally administrable soft-gel capsule comprising the suspension-based fill formulation of claim 1.

8. The soft-gel capsule of claim 7 wherein the soft-gel capsule comprises gelatin, plasticizer and purified water.

9. The soft-gel capsule of claim 7 wherein the plasticizer is sorbitol or a non-glycerin plasticizer.

10. A method of preparing a suspension-based fill formulation for soft-gel capsule, comprising the steps of:
(i) preparing a mixture of medium chain triglyceride (MCT) and lecithin; and
(ii) adding ibuprofen lysine to the mixture,
wherein the ibuprofen lysine has a d₅₀ value of less than 15 μm and a d₉₀ value more than 10 μm.

11. The method of claim 10, further comprising the step of heating the mixture before step (ii), preferably to a temperature above 50° C.

12. The method of claim 10, wherein the suspension-based fill formulation is homogenised by adding a colloid mill.

13. (canceled)

14. A method for the treatment of a headache or migraine comprising the step of administering a therapeutically effective amount of the soft-gel capsule according to claim 7 to a subject in need thereof.
